(19) United States
(12) Patent Application Publication
(10) Pub. No.: US 2004/0006340 A1
(43) Pub. Date: Jan. 8, 2004

(54) Bipolar electrosurgical instrument for cutting, desiccating and sealing tissue

(75) Inventors: Scott T. Latterell, Minneapolis, MN (US); Douglas S. Wahnschaffe, Otsego, MN (US)

Correspondence Address: NIKOLAI & MERSEREAU, P.A., 900 SECOND AVENUE SOUTH, SUITE 820, MINNEAPOLIS, MN, 55402, US

(73) Assignee: Gyrus Medical, Inc., Maple Grove, MN (US)

(21) Appl. No.: 10/188,207

(22) Filed: Jul. 2, 2002

(51) Int. Cl. A61B 18/18

(52) U.S. Cl. 606/48

(57) ABSTRACT

Electrosurgical forceps are described that have jaws capable of being closed relative to one another, the jaws each supporting electrode structures especially shaped to enhance the ability of the instrument to desiccate/seal or cut tissue structures clamped between the opposed jaws. One of the opposed jaws has a generally arcuate cross-section with a raised central zone and the other electrode has a recess adapted to accommodate the raised central zone of the cooperating electrode. By appropriating the shaping the mating electrode surfaces, tissue structures placed between the jaws are stretched laterally as clamping occurs. The stretching action prevents bunching of the tissue and results in improved desiccation, sealing and cutting. A fine, uninsulated conductor disposed on the one jaw, but insulated from the electrode surface on that jaw, serves as a cutting electrode.

### BACKGROUND OF THE INVENTION

I. Field of the Invention

This invention relates generally to electrosurgical instruments, and more particularly to an improved forceps whose jaws are especially designed to facilitate selective cutting, desiccation and sealing of tissue structures without the need for an instrument exchange.

II. Discussion of the Prior Art

The prior art is replete with electrosurgical forceps for use in open and laparoscopic procedures to cut through tissue structures, desiccate the tissue and any blood vessels to stem bleeding and for creating a fluid-tight seal between tissue structures along the margins of a cut. The Stern et al. U.S. Pat. No. 5,443,463 describes a coagulating forceps for use in open procedures in which the cooperating faces of its opposed jaws are generally planar and support a plurality of electrodes on one jaw and temperature sensing elements on the opposed jaw. Cutting of tissue is by way of a sharp blade that is actuated following electrocoagulation on opposed sides of the cut line.

The Fineburg U.S. Pat. No. 5,458,598 describes an endoscopic cutting and coagulating device, which, like the Stern '463 device has opposed jaw members whose opposed jaws are generally identical, each having a U-shape defining a central slot and with generally planar, albeit serrated, mating faces. A mechanical, sharpened blade, when actuated, passes longitudinally through the central slot following coagulation on each side of the cut.

The Wrublewski et al. U.S. Pat. No. 6,174,309 describes an electrosurgical instrument designed to seal and cut tissue. Embodiments for open and endoscopic procedures are described. In each case, the mating faces of the forceps jaws are such that one has raised electrode surfaces straddling a resiliently mounted cutting blade and the other has a recess for receiving the raised electrode surface therein when the jaws are closed and an intermediate groove in which the resiliently mounted cutting blade may enter. Coagulation takes place when tissue is squeezed between the jaws and a suitable voltage is applied between the raised electrodes on one jaw and on the opposite jaw. Cutting takes place when the voltage is applied between the cutting blade and the jaw having the recesses.

The Rydell et al. U.S. Pat. No. 5,445,638 describes a device somewhat similar to the Fineburg et al. '598 patent described above. It, too, has jaws having planar mating surfaces.

In each of the above-described embodiments, tissue to be coagulated, desiccated is clamped between the jaws of the device and a voltage is applied to the jaws to cause an RF current to flow through the captured tissue to heat and vaporize the moisture in the tissue. Cutting then takes place as a separate step, either by applying a cut voltage to a movable, metal blade member relative to a cooperating jaw or by effecting movement of a sharp blade through the previously desiccated tissue.

### SUMMARY OF THE INVENTION

We have found that significantly improved cutting, sealing or desiccation can be achieved, when compared to prior art devices of which we are aware, by providing a forceps with non-planar mating jaw surfaces. By providing one jaw with a first electrode surface having a raised male profile and the opposite jaw with a correspondingly shaped second electrode surface defining a female recess, as tissue is being clamped therebetween, it is placed under tension and stretched slightly, which allows it to be more readily and uniformly heated when a voltage is applied between the electrodes on the opposed jaws. As a further feature of our invention, a cutting electrode is supported on the jaw having the electrode with the raised male profile, but is electrically isolated from that electrode. Switching means are then provided for applying a desiccating or sealing voltage between the first and second electrodes or a cutting voltage between the cutting electrode and the first and second electrode surfaces.

Other features and advantages of the invention will become apparent from the following detailed description of a preferred embodiment, especially when considered in conjunction with the accompanying drawings in which like numerals in the several views refer to corresponding parts.

### DESCRIPTION OF THE DRAWINGS

FIG. 1 is a side elevation view of an electrosurgical cutting and sealing forceps designed for use in open procedures;

FIG. 2 is a cross-sectional view taken along the line 2-2 in FIG. 1;

FIG. 3 is a cross-sectional view taken along the line 3-3 in FIG. 1;

FIG. 4 is a side elevational view of an electrosurgical forceps designed for use in laparoscopic procedures and having an electrode structure in accordance with the present invention;

FIG. 5 is a cross-sectional view taken along the line 5-5 in FIG. 4;

FIG. 6 is a partial view of the forceps jaws having an alternative placement for a cutting electrode; and

FIG. 7 is an electrical wiring diagram showing one way of switching a radio frequency electrosurgical generator to the electrodes of the forceps instruments of FIGS. 1 and 4.

### DESCRIPTION OF THE PREFERRED EMBODIMENT

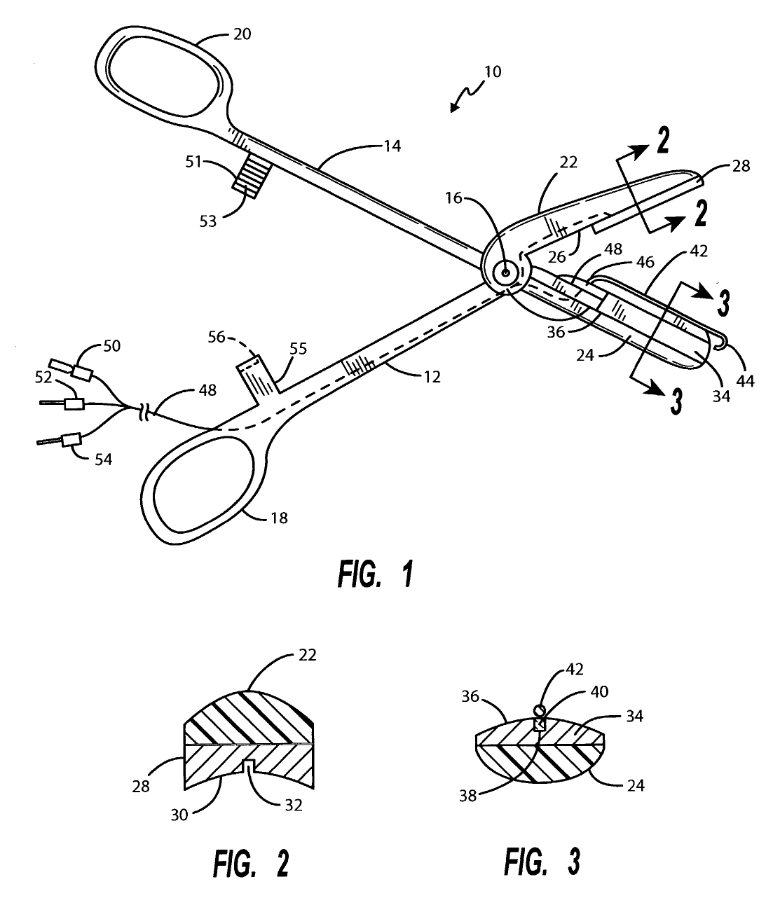

Referring to FIG. 1, there is indicated generally by numeral 10 a bipolar electrosurgical forceps that is adapted to clamp, seal, desiccate and cut tissue structures in the course of an open surgical procedure, the forceps 10 includes a first forceps half 12 and a second forceps half 14 that are electrically isolated from each other and pivotally joined by a fastener 16. The forceps halves 12 and 14 are preferably fabricated from a metal or plastic and have finger-receiving loops 18 and 20 at a proximal end thereof and jaws 22 and 24 at a distal end thereof.

Suitably fastened to a planar face 26 of the jaw 22 is a first conductive sealing electrode 28. Electrode 28 may be integral to the jaw 22. As can best be seen in the cross-sectional view of FIG. 2, the jaw/electrode 28 has an inwardly and upwardly sloping recess 30 whose sides converge to form a central, longitudinally extending notch 32 of rectangular cross-section. The exposed surfaces of the tapered recess and the notch are uninsulated.

The jaw 24 of the forceps half 14 has an electrode 34 either fastened to jaw surface 36 or integral with the jaw 24. As can best be seen from the cross-sectional view of FIG. 3, the electrode 34 may have a generally arcuate or beveled cross-section with a raised dome 36 in a central zone that is adapted to fit within the recess 30 of the electrode 28. A cut electrode 42 is mounted to and extends along the length of the electrode 34, and is isolated from electrode 34 by insulating plastic or ceramic 40. As can be seen from FIG. 3, the strip 40 is generally centrally disposed at the crown of the arcuate dome 36.

Referring once more to FIG. 1, a cutting electrode 42 may comprise a thin, rigid, isolated conductor disposed on the crown of dome 36 or, alternatively, may be a fine wire that is affixed at its distal end 44 to an end surface of the insulating strip 40. The other end 46 of the wire cut electrode 42 is set in an insulating plastic 48 on the jaw 24. Thus, while the cut electrode 42 runs closely parallel to the arcuate electrode 34 (typically within about 0.025 and 0.050 inch of electrode 34), it remains electrically insulated therefrom along its entire length. While a deposited conductor or a fine wire cut electrode has been found to function well, it is to be understood that the cut electrode can be otherwise configured so as to cooperate with the jaw members in the manner described.

Routed on or through the forceps half 18 is a three conductor cord 48 having terminals 50, 52 and 54 adapted to be plugged into jacks on an electrosurgical generator or a switch box associated therewith. A first of the three wires in the cord 48 connects to the electrode 28 mounted on the jaw 22. A second conductor in the cord 48 exits the scissors half 12 proximate the pivot fastener 16 and connects to the electrode 34. The third wire in the cord 48 connects to the cut electrode 42.

In operation, tissue to be sealed and desiccated is positioned between the open jaws 24 and 26 of the forceps instrument 10 and when the forceps halves 12 and 14 are brought together, the tissue becomes squeezed between the arcuate, domed, male electrode 34 affixed to the jaw 24 and the inclined walls or electrode 38 defining the recess 30. Squeezing the finger loops 18 and 20 toward one another results in the interposed tissue being squeezed and stretched by the wiping action between the mating electrode surfaces as the two are brought together.

By applying a predetermined voltage, via the cord 48, between the electrodes 28 and 34, tissue cells are desiccated and, in case the tissue structure is tubular, the walls thereof become sealed together. The notch 32 in the electrode 28 receives the cut electrode 42 therein, allowing the electrodes 28 and 34 to close tightly on the tissue structure to be electrocoagulated.

If it is desired to maintain the tissue structure clamped between the mating electrode surfaces for a time without the need for manually gripping the finger loops 18 and 20, there is provided a tab 51 on the forceps half 14 having a plurality of parallel, saw-tooth, detent grooves 53 formed therein. A cooperating tab 55 with a barb 56 on its undersurface is formed on the forceps half 12. As the forceps handles are brought together, the barb 56 can be made to fall into one of the plurality of saw-tooth notches 53 to thereby latch the forceps jaws in their closed disposition.

When it is desired to sever the tissue structure, a second predetermined voltage is applied, via the cord 48, between the cut electrode 42 and the electrodes 28 and 34. It will be recalled that the cut wire 42 and the electrode 34 are insulated from one another by virtue of the insulating strip 40 disposed in the groove 38 of the electrode 34. By placing the lower jaw 24 beneath the tissue structure to be cut, and draping it over the surface of electrode 34 so that the tissue is brought into contact with the cut electrode 42 and jaw 34 as a predetermined voltage is applied, the tissue will be severed.

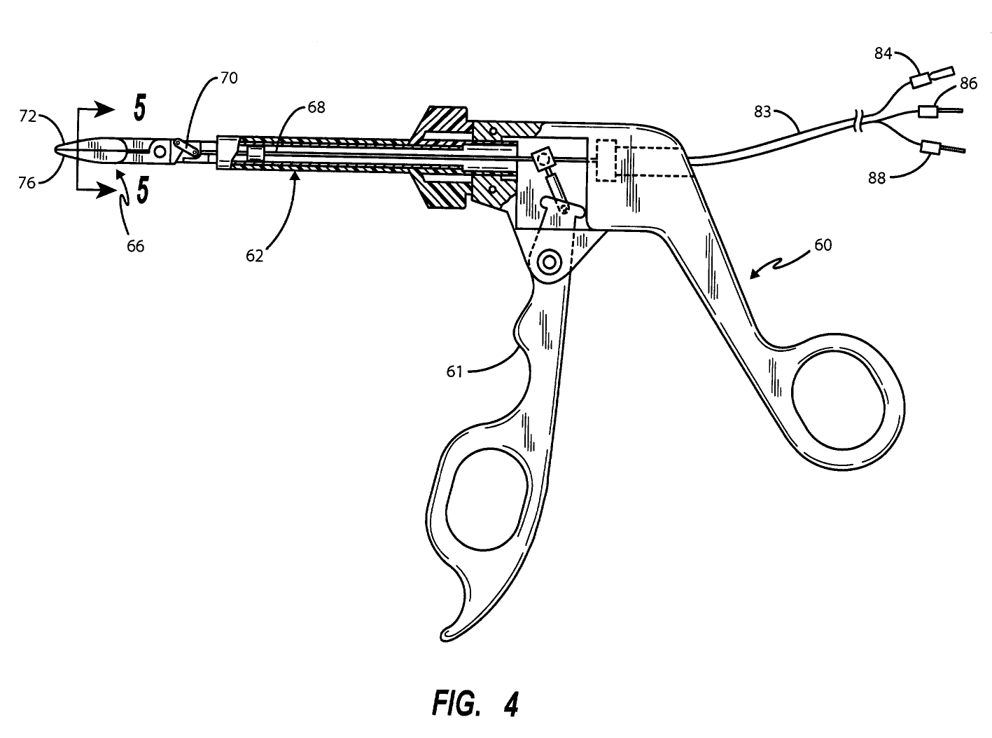

A second embodiment of the invention is illustrated in FIG. 4. Here, the forceps instrument is designed for a laparoscopic procedure. The forceps of FIG. 4 includes a handle member 60 that supports an elongated barrel 62 having forceps jaws at a distal end thereof, which are indicated generally by numeral 66. The handle and mechanism for opening and closing the forceps jaws 66 relative to one another may be like that described in the Rydell U.S. Pat. No. 5,462,546, the contents of which are hereby incorporated by reference as if fully set forth herein. As is described in that patent, by manipulating the scissors-like handle 60, a push rod 68 coupled to the jaws 66 by links 70 causes the jaws to open and close relative to one another. The first jaw 72 preferably comprises a rigid metal member having a concave recess 74 and a longitudinal notch 75, as shown in the cross-sectional view of FIG. 5, which is taken along the line 5-5 in FIG. 4. The jaws may be straight and aligned with the barrel 62 or they may be curved as in the Rydell '546 patent.

The other jaw 76 of the forceps comprises a metal electrode having a generally arcuate cross-sectional shape with a raised central dome that is adapted to fit within the concave recess 74 of the jaw member 72 when the jaws 72 and 76 are made to close relative to one another. The jaw 76 also supports a fine, narrow, conductive cut electrode 78 that remains electrically insulated from a metal jaw 76 in that its proximal and distal ends are embedded in an insulating strip 80 that is fitted into a groove 82 that extends longitudinally and is formed inwardly of the crest of the arcuate surface of the jaw 76.

It can be seen that when the handle member 61 is squeezed, the jaws 66 close relative to one another while the cut electrode 78 remains electrically isolated from conductive surfaces of the jaw 76. As with the embodiment of FIG. 1, because of the shape profile of the electrode surfaces of the jaw members 72 and 76, when tissue is disposed between the jaws and the handle member 61 is squeezed, the tissue will be draped over the convex arcuate profile of the jaw electrode 76 and stretched taut by wiping action of the concave surface of jaw 72 as the two come together. Now, by applying a predetermined voltage to appropriate ones of the jacks 84, 86 or 88, a current will be made to flow between the jaws 72 and 76 through the tissue captured therebetween to effect desiccation and/or sealing of the tissue structure. When it is desired to effect cutting of the tissue, a different voltage is applied between the cut electrode 78 and the jaw electrodes 76 and 72 to effectively cut through the tissue. It is not required that the jaws 72 and 76 be closed relative to one another during a cutting operation. By placing the lower jaw 76 beneath the tissue structure to be cut and draping it over the cutting electrode 78 so that the tissue engages both the cutting electrode and the metal jaw 76, upon a slight lifting motion on the handle, application of a cutting voltage between the two will result in severing of the tissue.

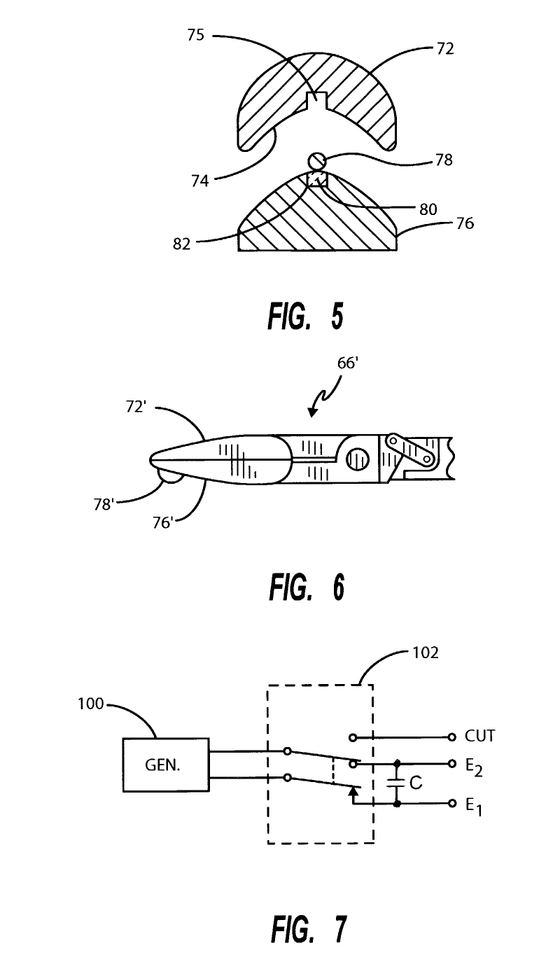

Referring next to FIG. 6, there is shown a partial view of the forceps jaw portion of the laparoscopic instrument illustrated in FIG. 4 but with the cutting electrode disposed on an exterior surface of a jaw rather than its mating surface. In this arrangement, the jaw assembly 66′ comprises a first jaw member 72′ and a second jaw member 76′ both being formed from a conductive material, such as stainless steel, Again, the blade assembly 66′ may be rectilinear or may have a curved profile. The mating faces of the jaws are preferably contoured in the fashion indicated in FIG. 5 to provide a stretching or tensioning of tissue structures as it is being pinched between the mating jaw faces. In the embodiment of FIG. 6, however, the cutting electrode 78′ is repositioned so as to be located on an outer surface of the jaw member 76′ with a layer of insulating ceramic effectively electrically isolating the cut electrode 78′ from the metal surface comprising the jaw member 76′. In the embodiment of FIG. 6, the cut electrode 78′ is a small bump or protuberance rather than a length of wire as in the embodiment of FIG. 5.

In the arrangements of FIGS. 4 and 6, an electrical cord 83, having three insulated conductors extends through the handle 60 and into the lumen of the tubular barrel 62 so as to electrically connect, individually, to the jaws 72 and 76 and to the cut electrode 78. Connector pins 84, 86 and 88 permit the forceps instrument to be connected to a power source such as a conventional electrosurgical generator.

FIG. 7 illustrates a switching arrangement for selectively coupling the outputs from an electrosurgical generator 100 to the three separate electrodes on the instruments of FIGS. 1, 4 and 6 to selectively desiccate/seal tissue structures or to sever such tissue structures. When the double pole, double throw switch on 102 is in the position illustrated in FIG. 7, the RF voltage from the generator will be applied between the electrodes 28 and 34 in the embodiment of FIG. 1 or electrodes 72/72′ and 76/76′ in the embodiment of FIGS. 4/6. When the switch 102 is thrown to its alternate position, a voltage for cutting tissue will be applied between the cut electrode 42 (FIG. 1) or 78 (FIG. 4) or the protuberance 78′ (FIG. 6) and the electrodes 34 (FIG. 1) and 76/76′ (FIGS. 4/6). It may prove expedient to connect a capacitor, C, between the conductors leading to the coag electrodes 28 and 34 in the embodiment of FIG. 1 or electrodes 72 and 76 in the embodiment of FIG. 4. The capacitor, preferably having a capacitance of about 2.2 nF functions to create an electrical link between the coag electrodes when the cutting electrode 42 (FIG. 1) or 78 (FIG. 5) is energized. However, when the coagulating electrodes 28 and 34 are energized, a sufficient voltage difference is generated between the coagulating electrodes to effect sealing/coagulation. The switch may take any number of forms and it, along with capacitor, C, may be conveniently located on the instrument itself, on the electrosurgical generator or on a foot switch module often used in electrosurgical systems.

With the jaw assembly 66′ on the laparoscopic instrument shown in FIG. 4 in place of the jaw assembly 66, sealing or desiccation takes place by closing the mating faces of the jaws 72′ and 76′ about the tissue and then applying a voltage between the two jaws causing a current flow through the captured tissue. When cutting, the voltage is preferably applied between the protuberance 78′ and the jaw members 76′. Then, by draping the tissue to be severed over the cut electrode 78′, it will also contact the jaw members 76′ functioning as a return electrode. Then by gently lifting the tissue while drawing the electrode 78′ thereacross, cutting occurs along a line traversed by the electrodes 78′.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

What is claimed is:

1. A bipolar electrosurgical instrument for clamping, sealing and cutting tissue comprising:
(a) a handle;
(b) a body joined to the handle;
(c) a jaw assembly joined to the body and arranged such that manipulation of the handle relative to the body allows tissue at a surgical site to be clamped between opposed jaws of the jaw assembly;
(d) a first of said opposed jaws having a first sealing electrode with a recess formed longitudinally along a length dimension of said first electrode; and
(e) the other of said opposed jaws having a second sealing electrode having a cross-section exhibiting a raised central zone adapted to conform to the recess of the first electrode for pinching and tensioning the tissue when the opposed jaws are made to close relative to one another and a cutting electrode supported by said other of said opposed jaws and being electrically isolated from the second sealing electrode.

2. The bipolar electrosurgical instrument of claim 1 wherein the cutting electrode extends in spaced, centered relation along a length dimension of a sealing surface of the second sealing electrode located above and generally parallel to said central zone.

3. The bipolar electrosurgical instrument of claim 1 wherein the cutting electrode comprises a protuberance disposed on and insulated from an outer surface of the second sealing electrode.

4. The bipolar electrosurgical instrument of claim 1 wherein the body is pivotally joined to the handle.

5. The bipolar electrosurgical instrument of claim 1 wherein the body comprises an elongated tube having a lumen extending the length thereof and the handle is mechanically coupled to the first and the other of the opposed jaws through the lumen.

6. The bipolar electrosurgical instrument of claim 1 and further including switching means for selectively applying a sealing voltage between the first and second electrodes or a cutting voltage between the cutting electrode and at least one of the first and the second sealing electrodes.

7. The bipolar electrosurgical instrument of claim 6 wherein the switching means is configured to apply a cutting voltage between the cutting electrode and both said first and second sealing electrodes.
